(19) United States
(12) Patent Application Publication
(10) Pub. No.: US 2020/0390622 A1
(43) Pub. Date: Dec. 17, 2020

(54) WALL-MOUNTED TOUCHSCREEN DIAPER VENDING MACHINE AND LOCATOR SYSTEM

(71) Applicant: Erin England, Dallas, TX (US)

(72) Inventor: Erin England, Dallas, TX (US)

(21) Appl. No.: 16/904,418

(22) Filed: Jun. 17, 2020

Related U.S. Application Data
(60) Provisional application No. 62/921,427, filed on Jun. 17, 2019.

(51) Int. Cl. A61F 15/00; A61F 13/551; G07F 9/02

(52) U.S. Cl. CPC A61F 15/001; A61F 2013/55195; G07F 9/0235; A61F 13/55105

(57) ABSTRACT

This invention involves a digital wall-mounted vending machine having helix discharge units which are used to store and dispense packaged objects such as a diaper changing kit and other diaper-changing products such as baby wipes. The diaper changing kit is comprised of a diaper, a changing table cover, and a disposal bag. The machine utilizes a horizontal helix dispensing system made of coils. The space between the coils can be adjusted to accommodate different sizes of products. A method is also provided to permit users to locate wall-mounted vending machines. The system involves a transceiver configured to communicate via a network. The transceiver may include a wireless transceiver and the network may include a cellular network.

### CROSS-REFERENCE TO RELATED APPLICATIONS

This patent application claims priority to U.S. Prov. Pat. App. Ser. No. 62/921,427, filed Jun. 17, 2019, which is hereby incorporated by reference for all purposes.

### BACKGROUND

### Technical Field

This invention relates generally to vending machines. More particularly, this invention relates to digital wall-mounted vending machines that dispense disposable diapers and related infant care and incontinent adult care accessories. This invention also relates to systems and methods for the dispensing of diaper kits containing a plurality of sizes of diapers and for the consumer's ability to locate vending machines via a mobile app.

### Background

Diapers are generally a necessity for very young children and incontinent adults. Individuals incapable of controlling the release of bodily waste in a manner sufficiently reliable to enable the use of restroom facilities, and those caring for such individuals, typically need to carry extra disposable diapers and changing accessories. This is typically because it is difficult to predict when a diaper worn by an individual may need to be changed. Caretakers of babies and others requiring diapers often carry spare, clean diapers for this reason. Many of the following discussions and examples are directed to use of diapers in infant care. However, it will be apparent that many of the same considerations apply to use of diapers for non-infant, incontinent persons. Storing and carrying clean diapers and related diaper changing accessories is often inconvenient. For instance, disposable diapers are generally rather bulky items not given to easy storage in a purse, pocket, or otherwise about the person. In addition, the problem can be exacerbated in that diapers are often needed in unpredictable circumstances when caregivers are away from home. In addition, it is often the case that it is not sufficient to simply change a soiled diaper and replace it with a clean diaper. For instance, cleaning of the individual who wore the diaper may need to be performed with diaper accessories, such as moist wipes. Also, it may be necessary to apply ointment or powder to the individual wearing the diaper to treat or prevent incidents of diaper rash or other skin conditions. Due to these considerations, caretakers of individuals requiring diapers often carry clean diapers and diaper changing accessories in a dedicated container, which is often referred to as a “diaper bag.” By doing so, when it becomes necessary to change a diaper, all of the materials needed by the caretaker are available in one location. While a conventional diaper bag provides a dedicated container in which diapers and diaper accessories can be stored and carried, the requirement to carry a diaper bag often adds to the difficulty of a caretaker's responsibility. For example, parents of young children often must carry a purse or bag for the parent's own needs. In addition to this, the parent may need to carry the young child; and, of course, the child's diaper bag. This can leave the parent with a burdensome amount of articles to carry and no free hands for other tasks.

There are currently very few options to purchase diapers and diaper changing accessories such as baby wipes in public (outside of grocery store and pharmacy). Diapers and wipes are not sold at most malls, airports, zoos, children's museums, parks, ballparks, amusement parks, and other kid-friendly destinations. This means that parents and caregivers with young children must take a diaper bag with them when traveling, running errands, and going to parks and restaurants with their children. The problem arises when the parent/caregiver is out in public and (a) forgets the diaper bag at home or in the car, (b) does not properly stock the diaper bag with clean, appropriately-sized diapers and baby wipes, or (c) the parent does not want the inconvenience of carrying the diaper bag due to its bulky nature. It is safe to say that a botched diaper change can very negatively impact a parent's day.

Children typically wear diapers up to age 3. There are 1,221,075 children under the age of 3 (diaper age) in the state of Texas alone. Young children can wear up to 12 diapers per day. Parents are incorporating their children into daily activities and travel plans now more than ever. Parents are also increasingly willing to pay a premium for convenience items when on the go. The ability to purchase diapers and wipes and hand sanitizer at the location of the public diaper changing station will help parents in a critical time of need.

Many attempts to address these problems have been made. For example, U.S. Pat. No. 6,723,080 issued to Habib discloses an enclosure containing a set of individually packaged sanitary accessories including prepackaged moist baby wipes, prepackaged instant hand sanitizer, baby liner, and a disposable diaper. U.S. Pat. No. 5,443,161 issued to Jonese discloses a baby changing kit with accessory items arranged within the enclosure to provide uniform and pliable surfaces for carrying and storage. The changing kit contains sanitary items for two complete diaper changes. U.S. Pat. No. 5,261,531 issued to Nieves discloses a feminine hygiene package comprising a disposable container enclosing a dry wipe, an enclosed wet wipe, and a sanitary napkin. U.S. Pat. No. 4,702,378 issued to Finkel discloses a disposable baby changing kit comprising of a sheet of plastic film folded in such a way to provide a number of pockets receiving various hygiene accessories. U.S. Pat. No. 4,964,859 issued to Feldman discloses a diaper with integral changing pad/disposable container wherein the changing pad is mounted on the outer surface of the diaper.

Nevertheless, each one of these references suffers from one or more of the following disadvantages. Most kits are bulky and therefore require a large vending machine. Others are packaged in a soft-sided package making them difficult to vend from a vending machine. Additionally, many of the kits require compression of the diapers, increasing the expense of manufacturing and packaging. Furthermore, none of the prior art kits are configured for vending multiple different sized diapers from a single sized package utilizing a low-profile wall mounted vending machine.

The current state of vending machines is inadequate to address this need. Of the machines that are in the marketplace today, they are often located in an inconvenient location (e.g., near a mall playground or in the middle of an airport terminal) due to their size and are coin-operated when most people today do not carry cash, let alone coins. Also, coin-operation is a deficient means of operating a vending machine because when a vending machine holds actual currency it is more susceptible to theft. The machines are also few and far between and difficult to locate. In addition, a vending machine may be provided to dispense fresh disposable diapers, however, such apparatus is bulky and because of this, many toilets are unable to provide it because of already limited space. Vending machines are often not wall mounted, but rather large, stand-alone machines too large to be located in a public restroom. The public restroom, specifically near the changing table in a public restroom, is the most convenient location for a diaper vending machine because that is where the caregiver performing a diaper change will need to procure a diaper. For parents on the go, the most convenient location for a diaper vending machine is a machine mounted on the wall near the changing table in a public restroom.

Diaper vending machines are known. For example, U.S. Pat. No. 7,188,748, which is hereby incorporated by reference, describes a vacuum-packed diaper vending machine. However, such machines vend volumetrically-reduced articles (i.e., vacuum-packed), which limits the different types of items that can be vended in the machines. Although prepackaged diaper kits are also known, they have not been engineered to properly vend from a wall-mounted vending machine in, for example, a public restroom. Rather, the aforementioned machines require posterior restocking of items, requiring a large hole to be cut into the wall on which the machine is mounted.

Digital vending machines with touchscreen displays are known. Vending machines equipped with transceivers configured to communicate via a wireless network and identify the location of vending machines and order a group of products as a single request are known. For example, U.S. Pat. No. 9,898,884, which is hereby incorporated by reference, describes a vending machine allowing purchase of items using a cell phone. However, such invention fails to be used for the dispensing of diapers and related diaper changing accessories.

Advertising facilitated by vending machines is known. The ability to remotely manage vending machine inventory and usage and to be alerted remotely to vending machine malfunctions and maintenance issues via a cellular network are also known. For example, U.S. Pub. Pat. App. No. 2014/0179231, which is hereby incorporated by reference, describes advertisements or coupons facilitated by the smart vending machine. Such advertisements and coupons are delivered to the user's cell phone subsequent to the user's use of the vending machine. The advertisements are targeted to the particular user based on purchasing history. Such method of advertisement is dependent on a person using the vending machine consenting to receive advertisements and using a vending machine in the same network at a later time to redeem the coupons. These methods require storing information related to the machine user and making product suggestions based on the user's previous vending machine usage.

Thus, there is a need for an innovative technology that can make disposable diapers and related items and accessories available where they are needed most.

### SUMMARY OF THE INVENTION

The present invention is a diaper vending machine system and method. In various embodiments, the system comprises a wall-mounted vending machine having a digital touchscreen and the ability to accept non-cash payments and an interactive mobile app to facilitate locating the machine and communicating with the system. In various embodiments, the vending machine may sell diapers and diaper changing products and be sized to be mounted on the wall near the changing table in public restrooms. In accordance with another aspect of an embodiment of the invention, a method of packaging a diaper kit is provided to facilitate the size and location of the vending machine.

In various embodiments, the vending machine may feature scrolling advertisements, for example, on the top portion of the screen such that the advertisements will be visible by not only the users of the machine, but also other people in the vicinity of the machine. Restrooms in particular are an excellent place to catch a captive audience. The advertisements may offer discount codes for childcare products, promote a local kid-friendly venue or resource, and so on.

The above summary of the invention is not intended to represent each embodiment or every aspect of the present invention. Particular embodiments may include one, some, or none of the listed advantages. Additional features and advantages of the invention will be apparent from the detailed description which follows, taken in conjunction with the accompanying drawings, which together illustrate, by way of example, features of the invention.

### BRIEF DESCRIPTION OF THE DRAWINGS

A more complete understanding of the method and apparatus of the present invention may be obtained by reference to the following Detailed Description when taken in conjunction with the accompanying Drawings wherein:

FIG. 1 illustrates an embodiment of a low profile vending machine;

FIG. 2 illustrates an embodiment of a product tray having a helix therein;

FIG. 3 illustrates an embodiment of a front cover of a low profile vending machine;

FIGS. 4a and 4b illustrate embodiments of a paper box;

FIG. 5 illustrates an embodiment of a diaper changing kit;

FIG. 6 illustrates a step in the assembly of a diaper changing kit;

FIG. 7 illustrates an embodiment of a diaper changing kit;

FIG. 8 illustrates embodiments of two diaper changing kits; and

FIGS. 9a and 9b illustrate embodiments of a wall-mounted vending machine.

### DETAILED DESCRIPTION

The present invention is directed towards systems and methods for providing diaper vending machines. In various embodiments, the diaper vending machines may be sized to be wall mounted in a public restroom at a location near a changing table and the diapers and other accessories may be packaged in a manner that facilitates dispensing from the wall-mounted machine. The diaper vending machine is configured to offer a wide range of products while occupying a limited volume so that it can be situated in areas where space is restricted.

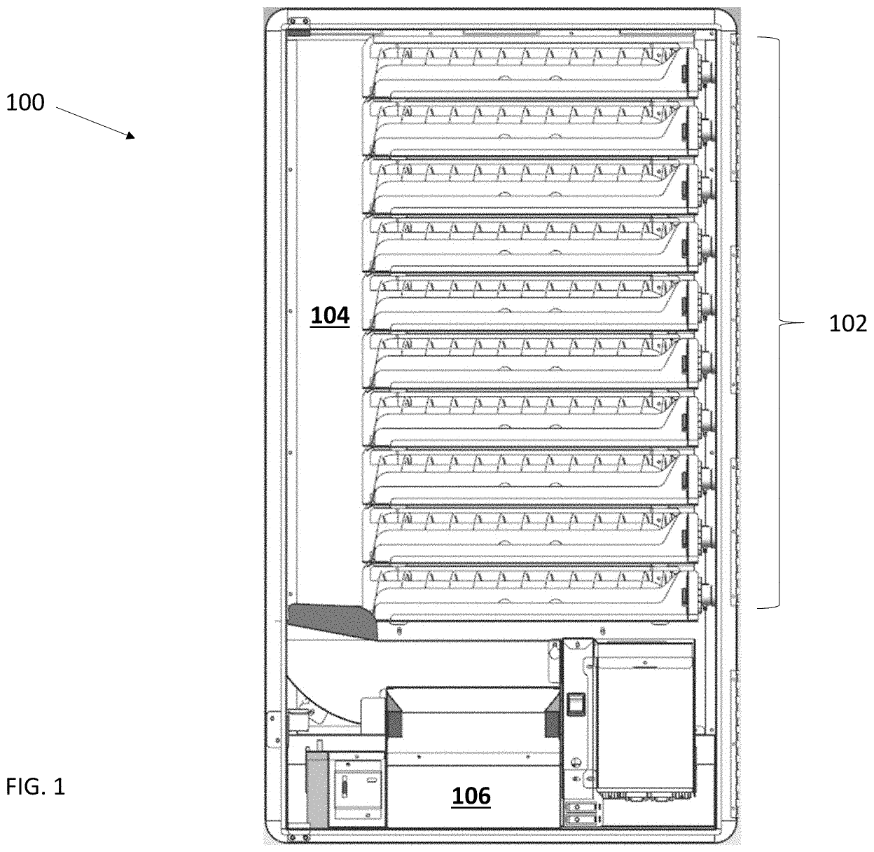

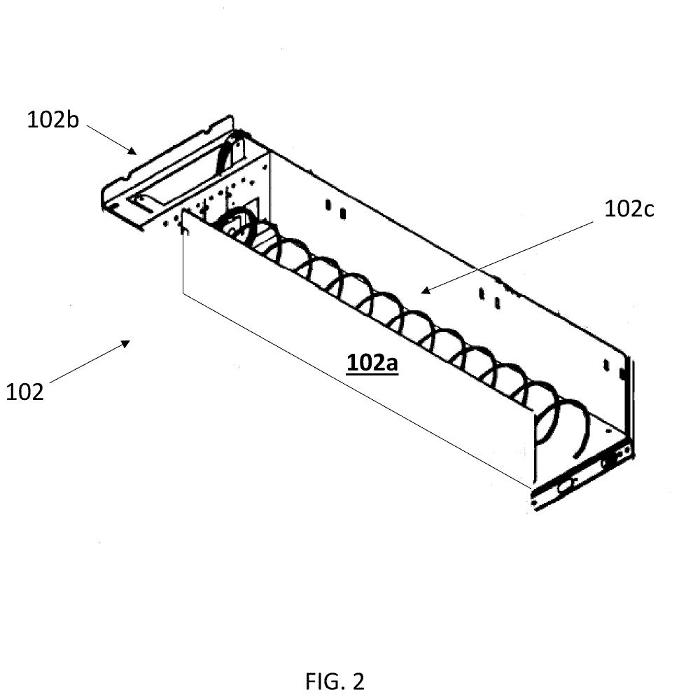

Referring now to FIG. 1, an internal view of an embodiment of a vending machine 100 having helix discharge units which are used to store and dispense packaged objects such as diapers and accessories, is shown. In the embodiment shown, the vending machine 100 holds a plurality of rows of products and dispenses the products one at a time from a side of a selected row. The end of each row of products leads to a chute 104 having an opening 106 at the bottom through which the product can be retrieved from the vending machine 100. Once a product is dispensed, the product falls down the chute 104 under the influence of gravity. In various embodiments, the machine preferably utilizes a plurality of rotatable helix dispensing spindles 102 oriented such that rotating the helixes moves objects laterally towards a vertical drop chute 104. In the embodied shown, the vending machine 100 has a plurality of helix discharge units 102 disposed laterally in a vertical relationship within the storage compartment portion of the machine 100. The storage compartment includes a front door (not shown). To the side of the helix spindles 102 is a discharge chute 104 and opening 106 positioned at the lower portion of the chute 104. The helix spindle is configured to retain all but the next-to-be-dispensed item. The spiral is capable of rotating through a position where it engages the items in the stack and pushes the next-to-be-dispensed item past the edge of the chute so that it falls off the edge and is then dispensed from a lower opening. A plurality of packaged items (not shown) may be positioned within each helix discharge unit 102. The discharge units 102 are designed so that they can be refilled when the front door is opened. Referring now to FIG. 2, each of the units 102 preferably comprises a tray assembly 102a, a drive unit 102b, and a helix 102c. In various embodiments, the helix units 102 are a rear driven helix which means that there is no positive connection between the drive unit 102b and the leading edge of the helix 102c, and the only power applied to the helix 102c is at its connection to the drive unit 102b. This allows the helix 102c to be turned and the packaged items to fall down the drop chute 104 when they reach the edge. When the drive unit 102b is energized, a drive shaft rotates the drive rod and consequently the helix 102c is rotated to propel the packages stored within the tray assembly 102a toward the drop chute 104 of the machine 100. The outermost package is propelled out of the tray assembly 102a into the discharge chute 104. The unit may be set up so that a package is discharged with each 360° rotation of the helix 102c.

Vending machines impose various limitations on the size, shape, weight, etc. of products which they are capable of vending. In general terms, products which are suitable for vending are those having length, width and thickness dimensions that will allow the individual product units to be stored, securely positioned, and selectively retrieved via mechanical or electromechanical means within the machine. The diaper changing kits described herein are configured to be vendable from a low profile, wall-mounted vending machine. In particular, the kits, or other products, loaded into the tray assembly 102a must be sized: (1) to have a thickness that fits freely between the augers; (2) to have a width that fits between the side walls of the tray assembly 102a; and (3) to have a height that fits under the bottom of a tray assembly (or top of the machine) disposed over the product. Oversized items or packages forced into the spaces will cause the machine to misvend. Additionally, in order to vend properly, the size of the item being vended must be larger than the diameter of the auger being used. Undersized items could cause vend problems. If the product does not fit the auger properly, a different auger must be used or the size of the package must be changed. The shape, size and thickness of the product affects how well it will fall off the tray. If vending problems occur, additional changes may be needed, such as adding product pushers or retiming the auger. In addition, any time a different sized package is loaded into a tray assembly 102a, the tray must be reconfigured and retested to ensure correct vending. The trays or shelves are mounted within the housing in such a manner that their height may be adjusted in small increments so that a wide range of packages can be accommodated. It also allows a number of shelves to be adjusted to hold the same product, or each hold a different sized package, for example. The products to be dispensed from this machine are relatively light and supplied in rigid packets. The upper surfaces of the shelves are preferably designed to be smooth and to minimize any friction with the products.

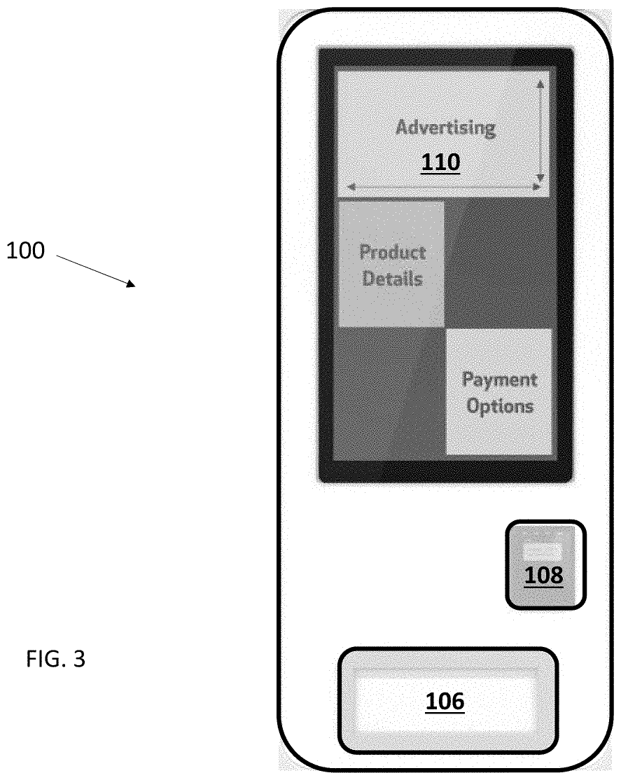

Referring to FIG. 3, there is shown a dispensing machine 100 such as a vending machine configured to mount to a wall. Machine 100 includes a body having an access door pivotably coupled therewith. The door can be securely locked to the body of the housing. Vending machine 100 is configured to dispense products on demand one at a time to a consumer, typically in response to payment by the user. A currency receiver 108 may be coupled with the door, for example, for receiving coins and the like and responsively generating a dispensation request for dispensing products from the machine 100. In addition to or as an alternative to receiving cash, currency receiver 108 may be configured for electronically reading consumer credit card information and/or receiving electronic and/or mobile payments. An access opening 106 is further provided, allowing a consumer to retrieve a dispensed product. The vending machine may include a digital display on a front panel thereof approximately 24 inches (diagonal). The display may be an LED-backlit display having a 16:9 aspect ratio and full HD (approximately 1920×1080 pixels). The dispenser may include, for example, a 7 inch touchscreen to display products and inventory status. The vending machine may include audio capabilities providing stereo sound with remotely adjustable volume. In various embodiments, the vending machine is a “smart” vending machines capable of communicating with a central cloud server to allow monitoring and managing from anywhere in the world. The vending machine may include software, cable hookups, and antenna to provide a plurality of connectivity options, including Ethernet, WiFi, and cellular (e.g., 3G, 4G, etc.) to facilitate monitoring inventory levels online. Customers expect a seamless high tech interface, similar to their cell phones or tablets. The smart vending machine may allow management of multimedia interactivity, digital signage, telemetry, inventory, social networking, and other features to increase revenue by reducing operating costs and elevating the customer experience. The vending machine may include software to facilitate remote sales audit data using, for example, VendLive, and may provide remote notifications of various items, such as Low Stock Alerts, Product Sale Alerts, Error Messages, and others, and may allow remote actions to be taken, such as Product Vend, Price Configuration, Software/Firmware Updates, Advertisement Management, and others. Additional details of implementing smart vending machines are described in, for example, U.S. Pat. No. 8,998,082, which is incorporated herein by reference. In some embodiments, the smart vending machine may allow for Social Vending, such as Twitter Social Vend, Facebook, Instagram, and SMS Enabled. As the machine is always connected, users can interact with it in other ways. Users can tweet the machine, for example, to receive a discount, or use innovative technologies such as facial recognition or emotion detection. Additional details of implementing social media capabilities into a smart vending machine and a location app for mobile devices are described in, for example, U.S. Pat. Nos. 9,721,060 and 9,898,884, both of which are incorporated herein by reference. The vending machine may accept a plurality of payment methods, such as EMV compliant, contactless only or 3-in-1, GBP, EUR, USD, and other currencies, Visa, Mastercard, AMEX, PayPal, ApplePay, AndroidPay, and others. Alternative payment methods can be implemented, such as an SMS gateway or a web portal, providing the ability for subscription purchases and more. A bill validator may be included for most currencies and/or the ability for cashless transactions, using, for example, a Nayax VPOS Touch to accept cards and NFC phone payments and/or QR/RFID scanner and validate ID. In various embodiments, the wall-mount dispenser is capable of being installed to an external surface of a wall without, for example, cutting a hole in the wall to form a recess. In various embodiments, screw holes and/or brackets mounted to the wall are the only defacement of the wall required to mount the low-profile vending machine. The vending machine may have a discreet design, which is sleek and elegant so that it will not look out of place and low profile so that it does not require much space, making it convenient for small bathrooms and cluttered locations. In some embodiments, the dispenser may be installed in an ADA compliant manner, such as, for example, the top of the screen being, for example, 48 inches off the ground to provide access to customers who use wheelchairs.

Wall-mounted vending machines are a compact, space-saving alternative to large floor standing vending machines. Wall vending machines can include many of the same features that are found in floor standing vending machines. In addition to restroom convenience products, they can also dispense a range of other products. In various embodiments, the vending machine has dimensions on the order of 39.4 inches (height)×17.5 inches (width)×11 inches (depth) and weighs (machine only) approximately 84 pounds. In other embodiments, the vending machine may be 23.6 inches wide×21.5 inches high×7.8 inches deep. In some embodiments, the vending machine may be housed in a cabinet that has overall dimensions of 1.5 m high (59 inches), 0.5 m wide (19.6 inches) and 0.5 m deep (19.6 inches) or larger. In some embodiments, the dispenser may include five or more adjustable product trays. In various embodiments, the vending machine may be able to dispense a plurality of separate products (depending on product sizes/configuration) with a column height of, for example, up to 13 inches (330 mm) (depending on product sizes/configuration) and capable of vending products having dimensions of, for example, 8.3 inches (210 mm) in height×5.1 inches (130 mm) in width×3.1 inches (80 mm) in depth. In other embodiments, typical ranges include products with heights in the range of 20 mm to 500 mm, widths in the range of 20 mm to 200 mm, and depths in the range of 5 mm to 100 mm. However, to be vendable from a low-profile wall-mounted vending machine, the width of the packaging much be less than the depth of the internal storage area and, in particular, the width of the trays of the vending machine.

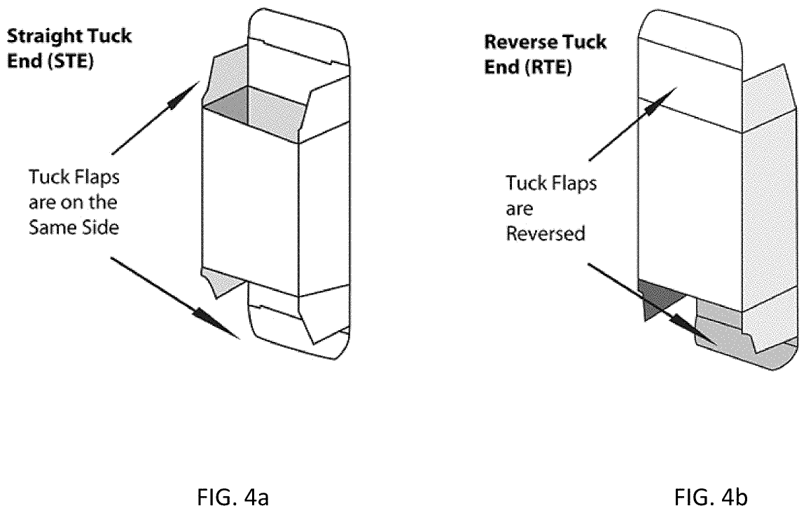

Turning now to FIGS. 4a and 4b, there is shown two configurations of a paper box for packaging the diaper kit according to an embodiment. By utilizing a standard sized box made of a stiff paper, more reliable ejection of products can be achieved by helping avoid off center forces on a product that might cause it to twist and potentially jam as it is ejected. In various embodiments, the paper box may be a straight tuck end paper box similar to the one shown in FIG. 4a or may be a reverse tuck end paper box similar to the one shown in FIG. 4b. The per-unit cost for the straight tuck end paper box is often higher than the reverse tuck end paper box because the reverse tuck end paper box wastes less paperboard during the die-cutting than the straight tuck end paper box. However, the bottom raw edge will show on the face of the product, making it less aesthetically pleasing than the straight tuck end box.

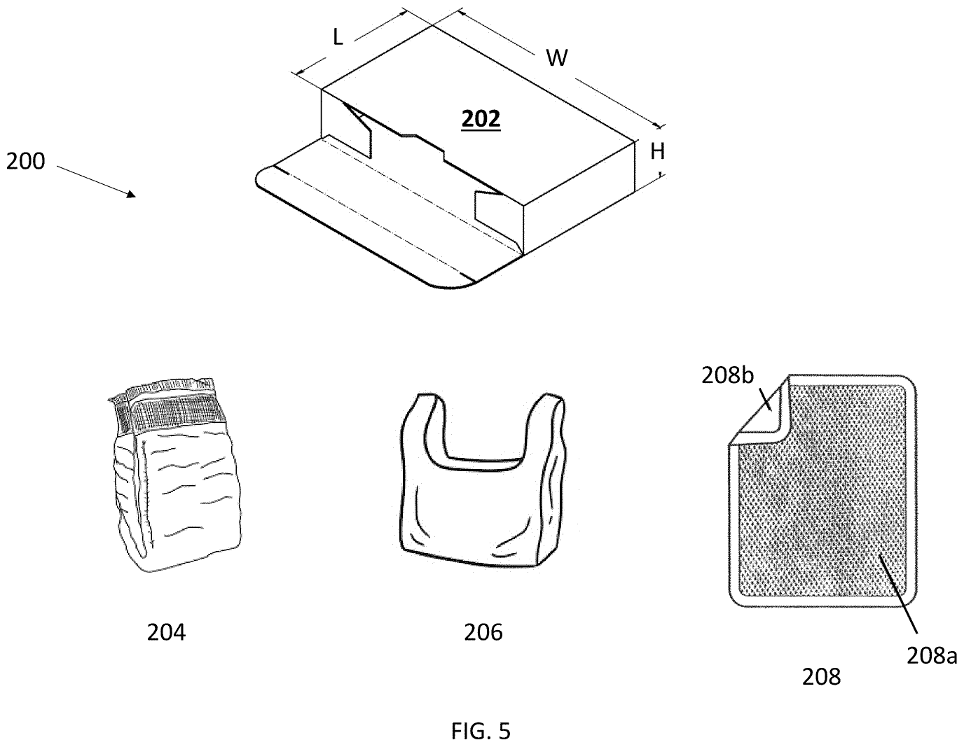

Turning now to FIG. 5, there is shown a disassembled diaper changing kit 200 according to an embodiment. The kit 200 may include a storage box 202, a diaper 204, a disposal bag 206, and a changing pad 208. The storage box 202 may be made from a relatively rigid material such as plastic or paper, and may include front and back panels having a length dimension L and a width dimension W. Between the front and back panels may be top and bottom panels and left and right panels to define the storage volume. In other embodiments, packaging outside of the storage box 202 may also be included, such as a plastic wrapper. In the embodiment shown, the diaper 204 is in a prefolded configuration typical of how diapers are packaged by the manufacturer, that is, a single fold on a midline thereof forming an outer surface of the diaper into a front outer surface and a back outer surface. In some embodiments, changing pad 208 consists of a prefolded rectangular piece, preferably in the range of eleven (11) to fifteen (15) inches in height and in the range of fifteen (15) to twenty four (24) inches in width and, in some embodiments, approximately thirteen and one half (13½) inches in height and approximately eighteen (18) inches in width. In some embodiments, changing pad 208 is preferably fabricated from a soft absorbent cloth-like material 208a on one side on which to place the newborn or infant, such as a 4-ply scrim material, with a water proof or water resistant coating 208b on another side. Changing pad 208 can be used, if needed, to clean the newborn or infant and can also be used to wrap up the wet or dirty diaper and other items to be discarded. Disposable plastic bag 206 is preferably fabricated as a small trash bag from a scented plastic material and may include outwardly extending arms which allow a dirty or wet diaper, as well as used moist baby wipe(s) and other packaging material, to be placed inside and the outwardly extending arms are then tied together. Disposable plastic bag 206 and its contents can then be discarded in any suitable trash receptacle.

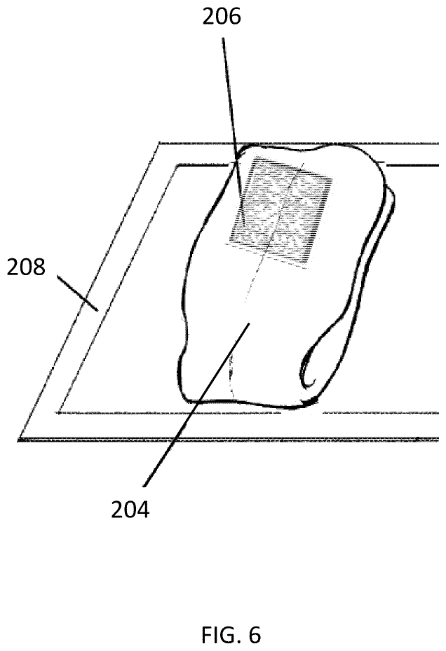

Referring now to FIG. 6, a disposable diaper 204, a bag for disposal of the soiled diaper 206, and a changing table cover 208 are shown. In the embodiment shown, the disposal bag 206 is folded into a small square shape and placed near an end of a prefolded diaper 204. The prefolded diaper 204 is then folded laterally along a midline of the front outer surface thereof over the disposal bag 206 to enclose the diaper 204 therearound. The folded diaper 204 is then placed on the table cover 208 and the changing table cover 208 is folded around the twice folded diaper 206. After the diaper 204 is folded and the cover 208 is wrapped therearound, the assembly, for a size 5 diaper, for example, is approximately 5 inches long, 3 inches wide, and 1.5 inches thick. The assembly is then slid inside the storage box or other packaging. The storage box is preferably dimensioned to be slightly larger than the assembly so it will fit inside thereof. As explained in more detail below, the storage box may be fabricated from a cardboard having sufficient weight to provide rigidity in order to accommodate a plurality of different sized diapers to allow the prepackaged diaper changing kit to be vended from a low-profile wall-mounted lateral vending machine located in facilities such as, for example, airports, shopping centers, gas stations, highway rest areas, family restaurants, sports facilities and arenas, amusement parts and children recreation centers.

To use the prepackaged diaper changing kit, the storage box or other exterior packaging is opened and the assembly is removed. The changing pad 208 is unfolded from around the disposable diaper 204 and placed on the surface where the newborn or infant is to be changed. The newborn or infant is placed on the changing pad 208 and the dirty or wet diaper is removed and placed into disposable plastic bag 206 and the new diaper 204 is placed on the child.

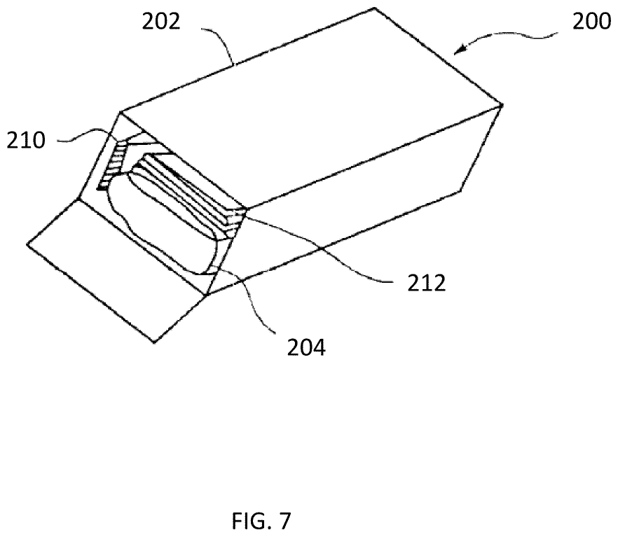

Turning to FIG. 7, there is shown a perspective view of an inside of an assembled diaper changing kit 200 comprising a plurality of items packaged inside the storage box 202. In the embodiment shown, a disposable diaper 204 folded over a disposal bag (not shown) and having a changing pad therearound can be seen. In addition, other items are also included within the storage box 202, such as ointment 210 and wet wipes 212. In other embodiments, the kit may include, in addition to or instead of the foregoing items, one or more prepackaged moist baby wipes, prepackaged instant hand sanitizer, prepackaged powder, and/or other items. In some embodiments, prepackaged moist baby wipes may be included in each prepackaged diaper changing kit or may be vended separately from the vending machine. Prepackaged moist baby wipes preferably have a rectangular configuration in the range of four (4) to eight (8) inches in height and in the range of six (6) to twelve (12) inches in width and most preferably are approximately seven (7) inches in height by approximately eight (8) inches in width and are folded to fit into an outer wrapper, preferably at least partially fabricated from a waterproof barrier material such as, for example, a foil or plastic material. The outer wrapper preferably provides a barrier to maintain the moisture level of the moist baby wipe packaged inside, but should also be relatively easy for the user to open to remove the moist baby wipe. The moist baby wipes are preferably fabricated from a porous material which is saturated with a hypoallergenic mildly fragrant cleansing lotion and are, most preferably, alcohol free. Prepackaged instant hand sanitizer is preferably prepackaged in a relatively flat waterproof packaging. Most preferably, prepackaged instant hand sanitizer consists of a prefolded sanitizing hand towel saturated with a sanitizing solution including a moisturizer, vitamins A and E and aloe therein. The prefolded sanitizing hand towel is approximately five (5) inches in height by approximately seven and one half (7½) in width when unfolded. Alternatively, prepackaged instant hand sanitizer could consist of a prepackaged portion of a hand sanitizing solution. Prepackaged instant hand sanitizer preferably contains no water, requires no towels and kills 99.9% of bacteria and germs. Other items could also be included in prepackaged diaper changing kit or vended separately, if desired, such as, for example, prepackaged baby powder, prepackaged diaper rash ointment, prepackaged teething reliever ointment, disposable gloves, disposable mask, and/or a disposable bib.

These items are preferably prepackaged together in a durable cardboard package having sufficient rigidity to allow vending from a vending machine. The durable cardboard package is configured to accommodate a prepackaged diaper changing kit for various sized diapers, for example, a small diaper for a newborn or infant and a large diaper for a toddler or older. In a preferred embodiment, at least two (2) different prepackaged diaper changing kits are anticipated each having a different size of disposable diaper, a smaller size being suitable for newborns and infants of up to approximately eighteen (18) pounds and a larger size suitable for infants and toddlers from approximately eighteen (18) pounds up to approximately thirty six (36) pounds. Other sizes for disposable diaper could also be used, for example, one size for newborns and infants of up to approximately twelve (12) pounds, a larger size suitable for infants from approximately twelve (12) pounds to approximately twenty four (24) pounds and yet a larger size suitable for infants and toddlers from approximately twenty four (24) pounds up to approximately thirty six (36) pounds. Still larger sizes for disposable diaper could be used in prepackaged diaper changing kit, if desired, for still larger children and/or adults.

In the embodiment shown, the box 202 is approximately 140 mm tall (appx. 5.512 inches), 40 mm wide (appx. 1.575 inches), and 80 mm deep (appx. 3.1496 inches) for a total volume of 448 mL (27.3386 cubic inches). In various embodiments, the vending machine may be stocked with disposable diapers of different sizes. In some embodiments, the horizontal rows of the vending machine may be adjustable to accommodate various product sizes, providing the ability to sell multiple distinct product lines and allowing different sized boxes to be utilized for different diaper sizes. However, in order to facilitate the dispensing of the diaper kits without the worry of the packages jamming or misvending, the dimensions of the box as well as the grade of paper of the box have been customized. In addition, in order to avoid reconfiguring the horizontal trays of the vending machine to accommodate different sized diapers, a single sized box may be utilized for multiple diaper sizes.

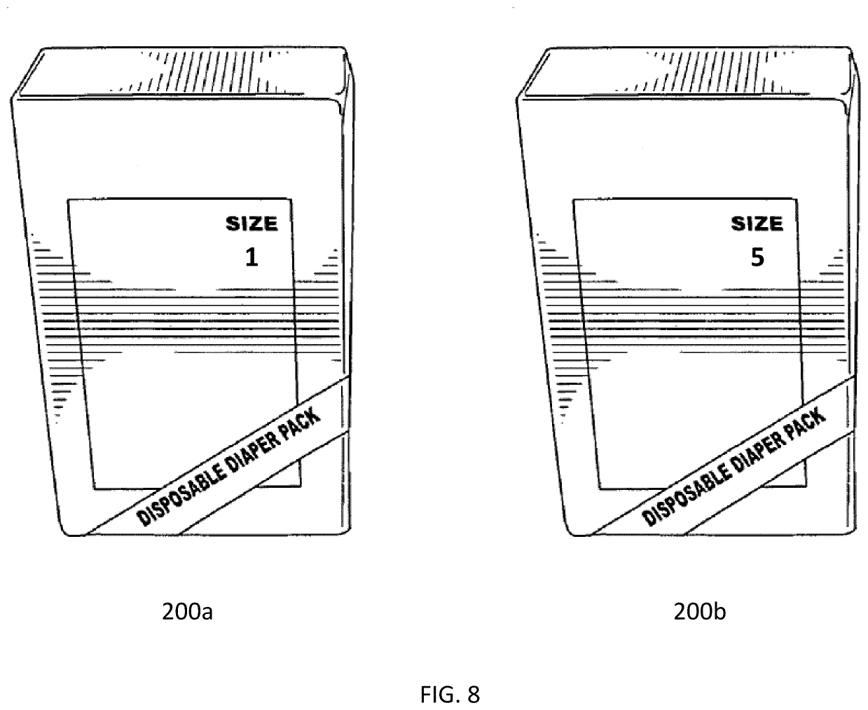

Turning now to FIG. 8, there is shown a perspective view of two assembled diaper changing kits 200a and 200b, one having a diaper assembly with a size 1 diaper therein and the other having a diaper assembly with a size 5 diaper therein. In general, size 1 diapers are smaller than size 5 diapers. For example, a size 1 diaper may have a length of less than 6 inches (in a prefolded configuration) whereas a size 5 diaper can have a length of more than 8 inches (prefolded). In addition, the widths and thicknesses of size 1 diapers are less than the widths and thicknesses of size 5 diapers. As another example, a size 2 diaper may be 7 inches long (prefolded) and 4.5 inches wide and have a thickness of 1 inch. When folded a second time as described herein, the length of the size 2 diaper may be reduced to approximately 4 inches with a width of approximately 5 inches. As another example, a size 6 diaper may be 9 inches long (prefolded) and 5 inches wide and have a thickness of 1 inch. In the embodiment shown, the storage boxes for the two kits are the same size. In some embodiments, the storage boxes for all the kits containing various sized diapers are the same size. By utilizing the same sized box, products can be substituted in the vending machine without requiring the spiral helixes and tray assemblies to be reconfigured. It should be appreciated that when the components are relatively large, such as a size 5 diaper, they will typically be sandwiched within the package tightly enough that little space will exist among the components and the packaging and that when the components are relatively small, such as a size 1 diaper, they will easily fit within the package and there will be extra room therein. Packaging articles in such a way that they are vendable presents various challenges. Many vending machines are configured to manipulate products having rigid surfaces and, when flexible items are vended, there is a risk of the article getting stuck in the machine. If, for example, the box is too small, the larger diaper may cause the box to bulge, which can cause problems with misvending. Similarly, if the box is not rigid enough, the box may bulge outwardly when packed with larger diapers and compress inwardly when packed with smaller diapers, which can similarly cause misvending. In terms of specific dimensions, the length of the storage box in one embodiment may be about five and one half inches in certain embodiments, the width may be about three and one half inches, and the thickness may be about one and one half inches. The specific dimensions have been discovered to be well suited for a vendable product configured for a wall-mounted low profile vending machine without requiring the diapers to be substantially compressed using, for example, a vacuum seal, which can damage the diapers.

In order to be vendable, the diaper changing kit needs to include a package consisting of a paper box with flexible articles packaged therein, wherein the paper box can be easily filled with a variety of different sized articles and subsequently closed and which is then shape-retaining. In various embodiments, the paper box for packing the flexible articles not only has good environmental qualities and sufficient strength, but also good printing qualities on its external surface so that an attractive package can be provided. In order to use a paper box to package flexible articles of various sizes and remain vendable, it is important that the paper box be sufficiently rigid. Paper of insufficient weight, when not supported by a rigid backing will easily get deformed, wrinkled and subsequently torn or punctured. This is also the case when flexible articles, like diapers, are packed in the paper box. One solution is to compress the flexible article, but this doesn't allow multiple sizes of flexible articles to be packaged in a single sized box. Another solution would be to provide a rigid backing for the paper. However, this increases the expense of the package and the complexity of the packaging process. In various embodiments, paper, cardboard, or combined layers of paper, having a total basis weight in the range of from 200 to 500 grams per square meter (gsm), are suitable. In some embodiments, the qualities of paper that present the most advantages from the environmental point of view for making the box are unbleached virgin papers or unbleached recycled papers. In order to be able to present an attractive package made with unbleached papers, it is, however, necessary to white-coat the outside of these papers to allow good quality printing. In a preferred embodiment, the flexible articles packed in the paper boxes are folded. In various embodiments, the paper used may be 350 g C1S (“coated one side”) white and have a thickness of 0.4 mm. In some embodiments, the thickness may range from 0.27 mm or less to 0.55 mm or more and have a grammage ranging from 210 gsm or less to 400 gsm or more. In some embodiments, the paper may be C2S (coated two sides), which may increase the grammage without significantly increasing the thickness.

The paper box utilized in various embodiments may be formed from various printed folding paper cartons, boxes made of the solid bleached substrate (SBS) paperboard, CCNB paperboard, kraft paperboard and/or specialty paperboard. The thickness of the paperboard in some embodiments is less than 0.8 mm in thickness. Bleached paperboard or SBS is a premium paperboard grade that can be single or multi-ply board. SBS is made from mostly hardwood, first-generation, chemically pulped fibers that are subsequently bleached, refined and formed. SBS is white on both sides and throughout the sheet. The relatively short hardwood fibers are coated and polished, creating an impressively smooth print surface that accommodates the highest quality imaging. Most SBS products are coated one-side only (C1S), but some may be partially or fully coated on the back side as well (C2S). SBS is not known for tear strength, but generally has good stiffness and compression strength.

Another possible material used in some embodiments is FBB (Folding BoxBoard) also called CTMP or TMP, which is multi-ply and utilizes a combination of chemical and some form of MP (Mechanical Pulp) or TMP (Thermo Mechanical Pulp). FBB is a light-weight board which uses fewer trees and offers yield advantages over SBS and CRB (Coated Recycled Board) and a significant weight savings. FBB comprises middle layers of mechanical pulp sandwiched between outer layers of chemical pulp. The top layer of chemical pulp is bleached and pigment coated. Being virgin fiber paperboard with consistent purity for product safety, the combination of inner layers of mechanical pulp with outer layers of chemical pulp creates a strong and stiff sheet, making it a low density material with high stiffness. The fully coated grades give great printing and visual impact. FBB is a low grammage solid board suited to the packaging of lightweight products, commonly available in 150-400 gsm. TMP is available in coated one side or coated two sides and is a cost alternative to SBS. The thickness of kraft board varies from 0.1 mm to 0.6 mm and the grammage can be 128-450 gsm.

Another important consideration is the stiffness or bending stiffness of the paper used in the packaging and the overall stiffness of the assembled box. After grammage and thickness, stiffness is the next most important property to consider when choosing paperboard. Stiffness is particularly important when determining the correct grade of paperboard for packaging applications. Without stiffness, paperboard would not be able to perform its primary function of providing the packaged contents with physical protection. Stiffness itself relates to other strength properties such as compression strength, toughness, creasability, foldability, etc. Stiffness play an important role in the distribution chain right through to the retailer and then the consumer. Maximum stiffness often needs to be achieved at the lowest possible grammage. Stiffness or bending stiffness is the degree to which paper or board resists bending when subjected to a bending force in its intended use. Bending stiffness is an expression of the rigidity of paper or paperboard and is often measured in Taber stiffness unit, in either gram-force centimeter (gfc) or milli-Newton meters (mNm). Since paperboard is an anisotropic material, measurements are made on strips cut in the machine direction (MD) and cross direction (CD) of the paperboard web. The stiffness ratio (MD/CD) gives an assessment of paperboard anisotropy. When specifying for a new end use or product, several factors must be taken into account. First are the weight and size of the product to be packaged. With cartons, it is only possible to consider the stiffness after the shape and dimensions have been decided. Not all paperboards of the same grammage or thickness have the same stiffness. Stiffness also has a major effect on the carton's resistance to bulging. The box compression strength requirement (physical protection) depends on a number of factors: the structural design of the carton, i.e. size and dimensions, supporting elements in the carton design, flap design, and loading direction, whether the contents support the package or not, types of secondary (transport) packaging, transport, storage methods and conditions (palletisation, stacking, climatic conditions), and material properties such as stiffness and compression strength of the paperboard. Structural designs and paperboard properties have to be matched to provide the required degree of performance from the packaging machine all the way through to the consumer. The carton must withstand various types of external loading and handling, protect the contents, be able to be vended from a low-profile vending machine, and reach the consumer without damage. For very small cartons, bulging of the panels is small or non-existent, which means that only the paperboard's compression strength is of importance (stiffness plays a minor role). In contrast, cartons with very large panels are much more dependent on stiffness than compression strength. In some embodiments, the paper board used for the storage box 202 has a thickness of 0.4 mm, a grammage of 350 gsm, and a stiffness of 18 to 31 mNm (MD) and 9 to 15 mNm (CD).

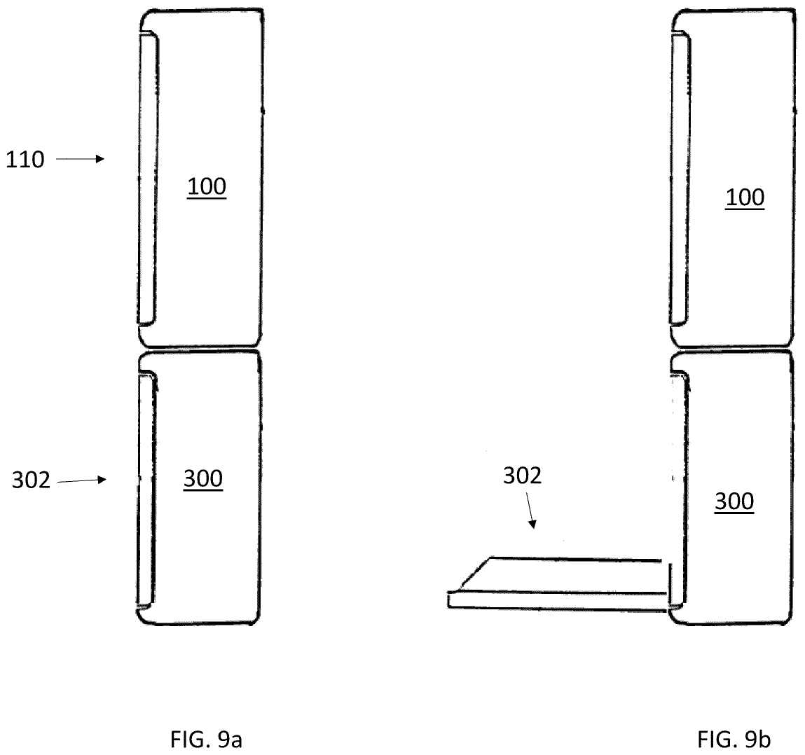

Referring now to FIGS. 9a and 9b, a diaper vending machine 100 is shown mounted to a wall (not shown) directly above a diaper changing station 300. The diaper changing station 300 may include a changing platform 302 pivotably mounted thereto so that it can be stowed away when not in use and can be lowered to provide a horizontal surface on which to change a baby's diaper. The display screen 110 of the diaper vending machine 100 is facing away from the wall, for example, approximately or slightly above the eye line of a person changing a baby place on the changing platform 302. In other embodiments, the diaper vending machine 100 may be mounted to a side of the changing station, on the same wall as the changing station, or on a different wall that the changing station.

Although various embodiments of the method and apparatus of the present invention have been illustrated in the accompanying Drawings and described in the foregoing Detailed Description, it will be understood that the invention is not limited to the embodiments disclosed, but is capable of numerous rearrangements, modifications, and substitutions without departing from the spirit and scope of the invention.

What is claimed is:

1. An arrangement at a commercial establishment for facilitating the changing of a diaper of a child by a customer of the commercial establishment, the arrangement comprising:
a baby changing station mounted on a wall and having a baby changing platform; and
a low-profile vending machine mounted on the wall for dispensing prepackaged diaper assembly kits, the vending machine comprising:
a housing having a rear wall, two opposing side walls, and a front door panel pivotally movable to permit access to an interior thereof;
a digital touchscreen display coupled to the front door panel and facing an exterior of the housing;
a vertical chute within the housing extending from an upper portion thereof to an opening at a lower portion thereof;
a plurality of horizontal trays mounted within the housing, each having a spiral guide driven by an electric motor for urging items towards the vertical chute, down which a dispensed prepackaged diaper assembly kit falls towards the opening; and
a plurality of prepackaged diaper changing kits disposed in a plurality of trays of the plurality of horizontal trays, each prepackaged diaper changing kit comprising a disposable diaper, a disposable plastic bag, and a baby changing liner inserted into a cardboard box;
wherein a first subset of the plurality of prepackaged diaper changing kits are disposed in a first tray of the plurality of horizontal trays and a second subset of the plurality of prepackaged diaper changing kits are disposed in a second tray of the plurality of horizontal trays;
wherein the disposable diapers of the first subset of the plurality of disposable diaper kits have a first size and the disposable diapers of the second subset of the plurality of disposable diaper kits have a second size, the first size being larger than the second size; and
wherein the cardboard boxes of the first and second subsets of the plurality of disposable diaper kits are the same size and are formed from paper having a thickness of at least 0.4 mm and a grammage of at least 350 grams per square meter.

2. The arrangement of claim 1, wherein the wall on which the vending machine is mounted is a wall of a public restroom of a commercial establishment.

3. The arrangement of claim 1, wherein the disposable diapers are prefolded disposable diapers and the prepackaged diaper changing kits are assembled by placing one of the disposal bags on an upper portion of one of the prefolded disposable diapers and folding the prefolded diaper therearound.

4. A method of dispensing disposable diapers, the method comprising:
providing a low-profile vending machine having a plurality of horizontal trays extending laterally within an interior thereof and terminating at a vertical chute, the interior of the low-profile vending machine being defined by a rear wall, four side walls, and a front door panel pivotally movable to permit access to the plurality of horizontal trays;
mounting the low-profile vending machine on a wall in close proximity to a diaper changing platform;
providing a plurality of disposable diaper kits, wherein each of the disposable diaper kits comprises a disposable diaper, a changing pad, and a disposal bag inserted into a cardboard box;
stocking a first tray of the plurality of horizontal trays with a first subset of the plurality of disposable diaper kits and stocking a second tray of the plurality of horizontal trays with a second subset of the plurality of disposable diaper kits;
wherein the disposable diapers of the first subset of the plurality of disposable diaper kits have a first size and the disposable diapers of the second subset of the plurality of disposable diaper kits have a second size, the first size being larger than the second size;
wherein the cardboard boxes of the first and second subsets of the plurality of disposable diaper kits are the same size; and
wherein the cardboard boxes are formed from paper having a thickness of at least 0.4 mm and a grammage of at least 350 grams per square meter.

5. The method of claim 4, wherein the plurality of disposable diaper kits are created by folding each of the disposable diaper, the changing pad, and the disposal bag at least twice.

6. The method of claim 4, wherein the disposable diaper, the changing pad, and the disposal bag are formed into a diaper assembly before being inserted into the cardboard box, the diaper assembly being created by placing the disposal bag on an upper portion of the disposable diaper in a prefolded configuration and folding the disposable diaper around the disposal bag and then wrapping the changing pad around the folded disposable diaper.

7. The method of claim 6 and further comprising:
wherein the diaper assemblies of the first subset of the plurality of disposable diaper kits have a height of approximately 5 inches, a width of approximately 3 inches, and a depth of approximately 1.5 inches; and
wherein the diaper assemblies of the second subset of the plurality of disposable diaper kits have a height of less than 4 inches, a width of less than 3 inches, and a depth of less than 1.5 inches.

8. The method of claim 4, wherein the cardboard boxes of the first and second subsets of the plurality of disposable diaper kits have a height of approximately 5.5 inches, a width of approximately 3.2 inches, and a depth of approximately 1.6 inches.

9. The method of claim 4, wherein the low-profile vending machine has a height of approximately 39 inches, a width of approximately 17.5 inches, and a depth of approximately 11 inches.

10. The method of claim 4, wherein the low-profile vending machine includes a point of sale credit card processor and a digital touchscreen display.

11. The method of claim 4 and further comprising:
stocking a third tray of the plurality of horizontal trays with one or more baby changing supply items selected from the group consisting of a prepackaged moist baby wipe, a prepackaged instant hand sanitizer, a prepackaged baby powder, and a prepackaged diaper rash ointment.

12. A low-profile diaper vending system comprising:
a low-profile vending machine comprising:
a housing having a rear wall, two opposing side walls, and a front door panel pivotally movable to permit access to an interior thereof;
a digital touchscreen display coupled to the front door panel and facing an exterior of the housing;
control and money handling circuitry for receiving a selection of an item to be released, confirming payment prior to initiating a release of the selected item, and displaying a plurality of advertisements on the digital touchscreen display;
a vertical chute within the housing extending from an upper portion thereof to an opening at a lower portion thereof; and
a plurality of horizontal trays mounted within the housing, each having a spiral guide driven by an electric motor for urging items towards the vertical chute, down which a dispensed item falls towards the opening;
a plurality of prepackaged diaper changing kits disposed in a plurality of trays of the plurality of horizontal trays, each prepackaged diaper changing kit comprising a disposable diaper, a disposable plastic bag, and a baby changing liner inserted into a cardboard box;
wherein a first subset of the plurality of prepackaged diaper changing kits are disposed in a first tray of the plurality of horizontal trays and a second subset of the plurality of prepackaged diaper changing kits are disposed in a second tray of the plurality of horizontal trays;
wherein the disposable diapers of the first subset of the plurality of disposable diaper kits have a first size and the disposable diapers of the second subset of the plurality of disposable diaper kits have a second size, the first size being larger than the second size; and
wherein the cardboard boxes of the first and second subsets of the plurality of disposable diaper kits are the same size and are formed from paper having a thickness of at least 0.4 mm and a grammage of at least 350 grams per square meter.

13. The system of claim 12, wherein the disposable diapers are prefolded disposable diapers and the prepackaged diaper changing kits are assembled by placing one of the disposal bags on an upper portion of one of the prefolded disposable diapers and folding the prefolded diaper therearound.

14. The system of claim 12, wherein the prepackaged diaper changing kits are specially designed so that the weight and size of the cardboard boxes facilitates proper vending using the spiral guides.

15. The system of claim 12, wherein the plurality of prepackaged diaper changing kits further comprises one or more baby changing supply items selected from the group consisting of a prepackaged moist baby wipe, a prepackaged instant hand sanitizer, a prepackaged baby powder, a prepackaged diaper rash ointment.

16. The system of claim 12, wherein the paper of the cardboard boxes has a machine direction stiffness of 18 to 31 mNm and a cross direction stiffness of 9 to 15 mNm.

17. The system of claim 12, wherein the advertisement displayed on the digital touchscreen display changes based at least in part on a selection of an item vended by the low-profile vending machine.

18. The system of claim 12 and further comprising a diaper dispenser location application for installation on a mobile device of a user to facilitate locating the low-profile vending machine.

19. The system of claim 18, wherein the diaper dispenser location application is configured to transmit to the user a location of the low-profile vending machine.
